(19) United States
(12) Patent Application Publication
(10) Pub. No.: US 2026/0327673 A1
(43) Pub. Date: Oct. 22, 2026

(54) Sampling Device and a Method for Collecting Microorganisms from the Environment by Chemotaxis

(71) Applicants: ETH Zurich, Zürich (CH); University of Technology, Sydney, Sydney, New South Wales (AU)

(72) Inventors: Roman Stocker, Zürich (CH); Estelle Clerc, Zürich (CH); Justin Seymour, Hornsby New South Wales (AU); Jean-Baptiste Raina, Glebe New South Wales (AU); Michael Arnold, Alten (CH); Christian Engler, St. Gallen (DE)

(21) Appl. No.: 19/104,422

(22) PCT Filed: Aug. 16, 2023
(86) PCT No.: PCT/EP2023/072593

§ 371 Date: Feb. 18, 2025

(30) Foreign Application Priority Data
Aug. 19, 2022 (EP) 22191181.1

(51) Int. Cl. C12Q 1/24

(52) U.S. Cl. CPC C12Q 1/24

(57) ABSTRACT

A sampling device for collecting microorganisms from aquatic environments by chemotaxis including a housing, at least one electronic control unit interacting with the housing/connected to the housing, and at least one rectangular cast that can be arranged within the housing—wherein the cast is designed to receive at least one assay plate, preferably multiple assay plates (eight or more); wherein the at least one assay plate comprises at least one well, preferably up to 20 or more wells, filled with at least one chemoattractant compound, and wherein each well is connected to the outside by at least a port; wherein the cast is further designed to receive at least one sealing plate at least partially covering the at least one assay plate, wherein the sealing plate is designed to be moveable along the at least one assay plate between a first position.

### CROSS-REFERENCE TO RELATED APPLICATIONS

This application is the United States national phase of International Patent Application No. PCT/EP2023/072593 filed Aug. 16, 2023, and claims priority to European Patent Application No. 22191181.1 filed Aug. 19, 2022, the disclosures of which are hereby incorporated by reference in their entireties.

### BACKGROUND

### Technical Field

The disclosure relates to a sampling device for collecting microorganisms from different environments, in particular aqueous environments, by chemotaxis and to a method using such sampling device.

### Technical Considerations

More than two million tons of sewage, industrial and agricultural waste is released every day into water bodies around the world (UN WWAP, 2003). This pollution is plaguing our environment as well as affecting animal and human health. The pollutants released in the environment are not only produced in high amounts in the industrial sector but are also directly derived from human waste. Their efficient treatment is problematic as most state-of-the-art disposal methods (incineration, landfill disposal, filtration and adsorption, chemical degradation) are either not sufficiently performant in capturing all pollutants from production sites or preventing them to enter the natural environment, or they are also often highly harmful to the environment by increasing atmospheric CO₂ concentrations, producing toxic by-products or directly affecting human and animal health.

The field of novel bacteria and enzymes discovery has been aiming at providing sustainable bio-based solutions to respond to this problem. However, biodiscovery encounters many fundamental biological and technical issues that hinders the success rate and application potential of novel findings, currently leaving biological interventions in pollution management as a promising yet unreliable solution.

The most traditional bacterial discovery methods in the environment usually consist in the collection of a large amount of material (e.g. water, soil) in an untargeted manner, bringing back the samples to the laboratory and treating them by multiple series of highly time-taking selective culturing steps and activity-based assays which often is biased for the growth of a limited number of microbial species (Eilers et al. Culturability and in situ abundance of pelagic bacteria from the North Sea. Applied and environmental microbiology, 66(7), 3044-3051; 2000).

More recent developments of advanced growth devices such as a micro-Petri dish (Ingham et al., The micro-Petri dish, a million-well growth chip for the culture and high-throughput screening of microorganisms. Proc. Natl. Acad. Sei. 104, 18217-18222, 2007), a boxed glass culture dish (Yunnan Inst. Tobacco Agri. Sci., CN112375687A), or the GALT Prospector™ system have nevertheless enabled isolation of novel microbes in recent years, yet remain laboratory-based techniques. The IChip (NovoBiotic Pharmaceuticals; Berdy et al., In situ cultivation of previously uncultivable microorganisms using the IChip. Nat. Protoc. 12, 2232-2242; 2017) is an in situ culturing technology that has been able to generate a large bacteria collection and discover novel antibiotics (W02014089053), but the starting bacterial inoculum remains unselective. Conversely, another screening device has been developed for in situ soil isolation but is restricted to oil-degrading strains (CN107118966A).

Advances in genomics and bioinformatics have also allowed computational targeted techniques such as genome mining to discover new enzymes in pooled or individual bacterial genomes (Ferrer et al., Mining enzymes from extreme environments. Curr. Opin. Microbiol. 10, 207-214, 2007; Ferrer et al., Metagenomices for mining new genetic resources of microbial communities. Microb. Physiol. 16, 109-123, 2009).

However, these technical and computational approaches are not only very time-taking or expensive (Popovic et al., Metagenomics as a tool for enzyme discovery: Hydrolytic enzymes from marine-related metagenomes. Advances in experimental medicine and biology, 883, 1-20. 2015) but are also oblivious of the natural bacterial dynamics happening in situ at the microscale (Stocker, Microbes see a sea of gradients. Science 338, 628-633, 2012) limiting the rate and outcome of novel biodiscovery as well as the probability of finding highly metabolically performant bacteria.

Individual microbial behaviors happening at the size of a single microbe, the microscale, control the biogeochemistry and productivity of natural ecosystems, and the rate at which each single microbes access substrates they need from their surroundings, depends upon the spatial distribution of substrates and the capacity of cells to exploit ephemeral nutrient hotspots (Azam, F. Microbial control of oceanic carbon flux: The plot thickens. Science 280, 694-696 (1998); Stocker, R., Marine Microbes See a Sea of Gradients. Science 338, 628-633 (2012); Clerc, et al., Survival in a sea of gradients: Bacterial and archaeal foraging in a heterogeneous ocean. in The Marine Microbiome (eds. Stal, L. J. & Cretoiu, M. S.) vol. 3, 47-102 (Springer International Publishing, 2022). These microscale behaviors and heterogeneity are tremendously important as they eventually impact large-scale geo-chemical processes, such as the carbon cycle. However, the immense resources and knowledge that microscale behaviors provide remain largely inaccessible by traditional bulk sampling. Our method overcomes this major challenge by accelerating biodiscovery through an unprecedented in situ screening technique for bacteria and enzymes discovery by leveraging the microscale bacterial behavior called chemotaxis, the ability of heterotrophic bacteria and archaea to move up or down chemical gradients and exploit transient nutrient patches in a heterogeneous environment.

The closest finding is an in situ device for microorganisms detection called Fungialter LTD (WO 2017/207756 A1 and WO 2019/154997 A1), acting as a pests control system in agriculture and horticulture. The device delivers an attractant into a growth substrate or water to direct responding microorganisms (i.e. fungi) to a detector for signalization of their presence. Fungialert aims to signal the presence of pathogens and can only target one compound at a time and is thus not suited for a high-throughput capture of microorganisms.

Conversely, chemotaxis appears to be widely used in microfluidic devices to investigate target species, moieties or compounds yet are limited to laboratory settings, such as in “Microfabricated Cellular Traps Based On Three-Dimensional Micro-Scale Geometries” (WO 2006/025858 A2).

### SUMMARY

An object underlying the proposed solution was therefore to overcome the existing hurdles and problems and to provide an approach to use chemotaxis for high-throughput bacterial capture, isolation and their further use in biodegradation and industrial processes.

This object has been solved by providing a sampling device with features as described herein and a method with features as described herein.

Accordingly, in non-limiting embodiments or aspects, provided is a sampling device for collecting microorganism from a liquid, in particular an aqueous environment by chemotaxis is provided, wherein the sampling device comprises:
a housing configured to receive at least one cast,
   wherein the at least one cast comprises means that are configured to receive and hold at least one assay plate, preferably multiple assay plates;
   wherein the at least one assay plate comprises at least one enclosed well, preferably up to 20 or more enclosed wells, filled with at least one chemoattractant compound, and preferably at least one water negative control (i.e. without or lacking) chemoattractant), and
   wherein each enclosed well comprises at least a port configured for fluid communication between the inside and the outside of the enclosed well;
   wherein the at least one cast is further configured to receive at least one sealing plate,
   wherein the sealing plate comprises at least one sealing element for sealing the port of at least one enclosed well of the assay plate,
   wherein the sealing plate is movably mounted on the cast such that it at least partially covers the at least one assay plate held by the cast,
   wherein said sealing plate is movable between
a first position, wherein the at least one sealing plate seals the port of the at least one well of the assay plate (sealing position/closed position), and
   a second position, wherein the at least one sealing plate opens the port of the at least one well of the assay plate (open position),

Thus, a sampling device is provided that allows screening for bacteria of interest based on their chemotactic preference to a substance in a wide range of environments through the use of specifically designed assay plates in combination with a specifically designed sealing plate. Cast and sealing plate are fixed or assembled inside the housing and upon assembling, allow access to remote environments.

The sealing plate prevents unwanted contamination during the traveling time of the sampling device through the water column by sealing the ports of the assay plate. At the target depth, the sealing plate opens for a predetermined amount of time and allows for the chemicals to diffuse and chemotaxis to occur, capturing responding bacteria inside the well of the assay plate. This is achieved by allowing the sealing plate to be moved between a first (closed) position when moving the sampling device through the aquatic environment and a second (open) position once the targeted position is reached. Once the sampling is completed, the sealing plate moves back from the second (open) position back to the first (closed) position for capturing the responsive microorganisms and moving the sampling device back to the starting point (i.e. forward-backward movement).

Electronics, secured on the side of the housing, allow for a fully stand-alone deployment. The housing possesses its own drop weight and needs to be actuated manually. However, other constructions may also be used. For example, a suitable construction could be based on a modified sampler or any other type of deployment mechanism. The present sampling device enables the implementation of a new biodiscovery method using chemotaxis that bacteria use to direct themselves in a targeted manner to chemical sources of preference. This allows for screening for the bacterial activity of interest and therefore accelerates the rate of biodiscovery, what traditional bulk sampling methods, advanced bacterial growth technologies and genome-based targeted enzyme discovery cannot do. Due to the optimized sampling device, it is now possible to sample environments that are usually difficult to access, opening new avenues for novel biodiscovery. Additionally, both of the applied microtechnology and sampling device are easily scalable and can reach a high-throughput efficacy therefore reducing the costs of the procedures and biodiscovery.

The present sampling device and method provide an approach for discovering new metabolically-potent bacteria in the environment and exploit them to degrade industrial and environmental pollutants, but not only. Other applications may include the discovery of bacteria and their enzymes that degrade, modify, transform or accumulate any type of chemicals both in industrial settings, or environments, the discovery of bacteria producing active substances of industrial or pharmaceutical uses, and/or the discovery of any other types of swimming microorganisms (phyto and zooplankton) with the above-mentioned abilities.

The present device and method enable a targeted search to a wide range of environments, including extreme ones, allowing to go beyond the current limit of bioprospecting and enabling the capture of bacteria and their enzymes with novel or enhanced metabolic activities.

Indeed, bacteria detected by applying the present device and method may be suitable to break down or transform any complex substances, which are often a characteristic of recalcitrant pollutants but not limited to oil, polychlorinated biphenyls (PCBs), polyfluoroalkyl substances (PFAS), pesticides, microplastics and more. Thus, bacteria and/or cocktails of their enzymes can be provided for targeted, cost-efficient and eco-friendly degradation of specific pollutants and other applications as mentioned above. This approach can efficiently address the degradation of harmful chemicals in industrial sources, increases performance of pollutants removal in wastewater treatment plants, prevents additional pollution to be leaked in the environment, and may allow to intervene directly in the environment in cases of emergency (i.e. oil or chemical spill).

The sampling device and sampling method have a broad range of applications in multiple industries, including pharmaceuticals, food, agriculture, chemicals, biotechnology, governmental, and more. By targeting a specific compound of mixture of compounds, it is possible to deliver highly performant single bacteria and/or enzymes or bacteria and/or enzymes cocktails derived from the specific bacterial consortium. It allows for bacteria and enzymes discovery on demand of customers and their specific needs, while providing subsequently custom single bacteria and/or enzymes or bacteria and/or enzymes cocktails.

The different features of the sampling device are now described in more detail.

### Cast

As described, the cast is configured to receive and hold the assay plate(s). The cast is designed to hold or keep at least one assay plate, preferably more than one assay plate, most preferably more than four, such as eight assay plates or more in its position.

In a non-limiting embodiment, the at least one cast is made of pressure and corrosion resistant material, in particular is made of polyoxymethylene (POM). Besides being a pressure and corrosion resistant material, the selected material does not release chemicals repulsing bacteria. However, any other inert material with the required properties may be suitable.

As mentioned, the cast has a rectangular, elongated shape. Other shapes are also applicable, such as square, diamond, cross and more. The size of the cast is chosen in a manner to deploy an optimal number of assay plates and avoid at the same time the system being too heavy.

In a non-limiting embodiment, the cast is designed in form of a frame with two (opposite) surface sides, wherein each surface side is configured to receive and to hold at least one assay plate.

In a non-limiting embodiment, the cast frame has two opposite long edge sides and two opposite short edge sides and two opposite surfaces. The frame edge sides are designed to create a (rectangular) space with two (rectangular, planar) opposite openings on both opposite frame surfaces. Such a cast frame can also be described as a (flat) rectangle wherein only two opposite surface sides are designed to receive and to hold the assay plate.

At least one surface side of the cast frame is designed to receive at least one sealing plate (e.g. can be covered by at least one sealing plate). In this case, the other surface side of the cast frame may be covered by any conventional means. In a non-limiting embodiment, both surface sides of the cast frame can be designed to receive each one at least one sealing plates. Thus, the cast frame allows for one or two sealing plates to be employed and actuated.

In a non-limiting embodiment, the cast has a (spatial) cubic shape with four surface sides (with openings), wherein each of the four surface sides is configured to receive and hold assay plates. Each surface side may be configured to receive at least one assay plate and up to three assay plates. Thus, such a cast may hold up to 12 assay plates at the same time. However, it is to be understood that depending on the shape and dimensions of the cast the number of plates is adjustable and can be even more than 12 assay plates.

Furthermore, such as (spatial) cubic cast is configured to receive up to four sealing plates (one sealing plate for each surface side) to be employed and actuated.

As mentioned, the cast is designed to receive and hold the assay plates. For this purpose, the cast may comprise a type of tracks and any other suitable means to hold the assay plates. A suitable mean to hold the assay plates may be a recess that is inserted into cast (e.g. the cast frame). The assay plate is placed into said recess. The plates are screwed in the cast (for example with two or more screws).

In a non-limiting embodiment, the cast is connected to at least one electronic control unit and to at least one motor, in particular a pressure compensated motor for moving the sealing plates between the first closed position and the second open position of the well ports of the assay plates. Control unit and motor allow for autonomous activation at depth (based on a sensor).

The speed of the motor should be chosen such not to create turbulence upon motion. The motor contained in the cast is fitted with a high reduction gear box to provide a slow seal closing movement in order to reduce turbulence and prevent disturbance of sampling collection.

In a non-limiting embodiment, motor and electronics can be combined in a single enclosure, for example a pressure compensated enclosure to reduce system complexity and external connections. Said enclosure may be attached to the cast, preferably such that cast and enclosure with motor and electronics can be handled as one piece. This would enable the use of a hermetically sealed external housing, since no cable feed-through would be required.

A combination of cast and enclosure with motor and electronics would fit into an openable housing, as described in detail further below.

### Assay Plates

The assay plates have been described previously (Lambert., et al. A microfluidics-based in situ chemotaxis assay to study the behaviour of aquatic microbial communities. Nat. Microbiol. 2, 1344-1349 (2017) and are called In Situ Chemotaxis Assays. By using this approach bacteria are attracted in situ based on their chemical preferences and captured directly in their natural environment for further laboratory culturing and characterization. Chemotactic responses can be quantified by counting bacteria present in the wells using flow cytometry, and the sampled bacteria can be further characterized by extracting their DNA, sequencing them and directly isolating them. The assay plates are made of a polymer material, such as PMDS or polycarbonate.

As mentioned, the at least one cast contains one or multiple assay plates. Each assay plate comprises at least one well, preferably up to 20 wells, filled with at least one chemoattractant compound and at least one negative control, such as ultrafiltered seawater. The number of wells of each assays plate can vary and is not limited to 20 wells. Thus, also the numbers of wells per row can vary and is not limited to five. The number of wells solely depends on the overall size of the assay plate and sampling device.

Each (circular) well of the assay plate is enclosed with the exception of a port that allows the fluid communication between the inside of each well with the outside environment. Each well has a diameter between 5 and 8 mm, preferably between 6 and 7 mm. The distance between the center of adjacent wells varies and can be between 9 and 17 mm. The depth of each well is between 2 and 4 mm, preferably 3 mm. It is to be understood that the dimensions of the assay plate can be modified and adapted according to the spatial requirements of the cast.

In a preferred embodiment, the port of each well of the at least one assay plate is offset from the center of the well. The port has a diameter between 700 and 900 μm, such as 800 μm. The port protrudes from the well. The length of the port protrusion is about 1.5-2 mm, preferably 1.6 mm-1.7 mm. The shortest distance between port and well circumference is 0.5-0.8 mm, preferably 0.6-0.7 mm, such as 0.65 mm.

The port allows the chemoattractant to diffuse out of the well into the environment, preferably an environment with an aqueous phase, such:
Marine ecosystems: including any depth of seas and oceans water column, mudflats, seagrass meadows, mangroves, intertidal zones, salts marshes, coral reefs, lagoons, estuaries hydrothermal vents, backwaters;
Freshwater ecosystems: including lakes, rivers, ponds, streams, springs, bogs, wetlands, marshes, snow and ice, glaciers and ice caps;
Soil and groundwater;
Industrial and man-made environments: including wastewater treatment plants, freshwater reserves, rainwater and stormwater retention tanks, bioreactors and chemical tanks, aquaria, swimming pools.

Thus, the sampling device may be deployed in any type of environment containing at least an aqueous phase.

Submersion of the device in water results in a chemical microplume extending for example 1-2 mm above each well within a preferable deployment time of, but not limited to, 1 to 4 h.

In turn, the microorganisms attracted by the selected chemoattractant(s) can enter the well through the port. The chemotactic response is subsequently quantified by counting bacteria present in the wells using flow cytometry, and further characterized by extracting and sequencing their DNA, and directly isolating them.

The assay plate device is made of inert materials, is single-use and is fabricated using a standard soft lithography workflow, based on a mould created using three-dimensional printing. The material used for the assay plate(s) may be glass, polydimethylsiloxane (PDMS) or polycarbonate. However, any other inert material, in particular inert plastic material with the required properties may be suitable.

### Sealing Plate

As described previously, at least one sealing plate is provided for at least partially covering the at least one assay plate. The at least one sealing plate is placed and mounted in a movable fashion on or to the cast, in particular on the cast frame. The at least one sealing plate is preferably mounted movably on one side of the cast, in particular on one surface side of the cast frame as previously described.

In a non-limiting embodiment, at least two sealing plates are provided on the opposite sides of the cast, in particular opposite surface sides of the cast frame, as previously described.

Said sealing plate is designed to move along or over the surface of the at least one assay plate between a first position, wherein the at least one sealing plate seals the port of the at least one well of the assay plate (sealing position/closed position), and a second position, wherein the at least one sealing plate opens the port of the at least one well of the assay plate (open position). The sealing plate is freely movable between both positions for allowing closed and open position of the assay plate whenever required.

The sealing plate seals the ports of each well of the assay plate with high precision and slides autonomously at the predetermined sampling depth to release the chemical plumes. Thus, the sealing plate prevents unwanted contamination of the wells of the assay plate during the traveling time through the water column by sealing said wells of the assay plate.

The overall design of the sealing plate including thickness and angles to avoid turbulences. The sealing plate may be designed in a grid-like manner with alternating slit-forming gaps for open position and bridges for sealed position. The width of the slits or gaps is chosen to enable optimal chemical diffusion and chemotaxis.

The bridges are provided with evenly spaced elements for sealing the port of the well. The sealing elements are arranged in a line on said bridges, which is parallel to each row of wells of the assay plate, when the sealing plate covers the assay plate.

The number of said sealing elements on each bridge is identical to the number of wells in each row of the assay plate. For example, if an assay plate has five wells per row then five sealing elements are provided. This ensures that each port of the wells of one row in the assay plate can efficiently be sealed.

The sealing elements may have a spheric shape and are embedded in holes within the sealing plate. Size and shape of the holes correspond to size and shape of the embedded sealing means. In a non-limiting embodiment the sealing means are provided in form of a rubber sphere. The rubber spheres are embedded in the plate and provide an efficient seal while in the closed position. In the closed position, the rubber spheres are positioned on top of the assay plate ports and seal the ports to prevent contamination and chemical leakage from the well. In the open position, the sealing plate is moved such that the rubber spheres open and expose the port of the wells to the external environment.

The at least one sealing plate may be made of the same material as the cast, i.e. a pressure and corrosion resistant material, in particular is made of titanium. Other types of material with the same required properties may be used.

The geometry of the sealing plate has been optimized for inducing the least amount of turbulence while sampling. The sealing elements (rubber spheres) are spherical and compressible, allowing for high-pressure sealing of the wells. The sealing plate does not only prevent contamination but also prevents the chemoattractants to leak before deployment and enables sampling precision. Furthermore, while the assay plate enables the collection of bacteria, the sealing plate enables controlled release of the chemoattractant.

### Housing

The housing is a pressure compensated enclosure, in particular a tubular housing, such as a modified Niskin bottle for hosting the sealing plate and cast and is purposed to maintain a low-flow environment during the deployment. The enclosure or bottle is also made to be connected to the electronics control unit, preferentially screwed to a frame attached to its side.

The housing is designed for obtaining samples of water at a specific depth. The housing is a tube, usually plastic to minimize contamination of the sample, and open to the water at both ends. Each end is equipped with a cap which is either spring-loaded or tensioned by an elastic rope. The bottle is closed by the action of a messenger weight that trips both caps shut and seal the tube.

A non-limiting embodiment of the housing uses actuated valves that may be either preset to trip at a specific depth detected by a pressure switch, or remotely controlled to do so via an electrical signal sent from the surface. This arrangement conveniently allows for a large number of housings to be mounted together in a circular frame termed a rosette.

The housing used in the present case was modified to be able to host the cast containing the assay plates and the sliding plates, and to favor its attachment to the electronic control unit on its side.

In a first modification, the clamping mechanism of the enclosure or bottle was modified to be able to keep the lids open while mounting the cast inside the housing. For this purpose, the closing mechanism of the bottle was modified, consisting of two plugs that are pulled away from the openings of the housing by two springs. Before deployment they are locked in an open position by a set of retaining wires, keeping the lids open under tension and ready to be activated by the triggering mechanism.

In an alternative development, the closing mechanism enabling the lids of the housing would be fully actuated electronically via mechanical arms, whereby the entire closing mechanism is controlled by an actuator. This would allow to control the speed, exact timing and closing strength of the lids.

In a further modification, a hole was drilled in the side of the bottle to host a cable gland, allowing for the connection of the cast to the external electronic control unit on the side of the housing.

### Electronic Control Unit

In a non-limiting embodiment, at least one electronic control unit is connected to the housing, for example secured on the side of the housing in a titanium enclosure allowing for a fully stand-alone deployment. However, the electronic unit may also be placed at any other suitable location.

The electronic control unit contains the electronics controller and the batteries. The controller is used to acuate the motor moving the sealing plate. The controller receives the deployment parameters from a removable SD card and starts the deployment sequence when the preset depth is reached. While the device is under water, the controller logs sensor data to a file on the SD card, containing the following parameters: date, time, water depth, position of the sealing plate, power supply voltage, motor current, attitude of the device and water temperature.

The controller is placed inside the watertight titanium enclosure and switched on by a watertight plug switch. The electronic control unit is connected to the motor contained in the cast within the housing, such as a modified Niskin bottle, by a watertight connector, that goes through a cable gland in the side of the housing.

The electronic control unit contains a battery pack (12 V to 24 V).

The removable external data container (SD card) allows the operator to quickly change the parameters of subsequent deployments after retrieval. The data container is fully watertight, and pressure rated to the diving depth of the system, enabling the handling of the data container without having to open the seals of the pressure housing of the controller. The data container also allows to record data during the deployment which can be retrieved and provides information about the deployment sequence and deployment environment (system power consumption, water temperature, depth, assay plate orientation). An external watertight indicator light, pressure-rated to the diving depth of the system, is mounted on the electronics control unit. Before and after deployment, this indicator is visible for the operator and provides information on the status of the system and deployment parameters by showing different colors and flashing sequences. An Inertial Motion Unit (IMU) is mounted on the system to provide information on the status of the assay plates during deployment.

The sampling device as described may be employed in a method for collecting bacteria from liquid environments by chemotaxis, wherein, in non-limiting embodiments or aspects, the method comprises:
Providing at least one assay plate with at least one well filled with at least one chemoattractant compound;
Placing the at least one assay plate in the at least one cast, wherein the at least one assay plate is at least partially covered by the at least one sealing plate such that the ports of the wells of the assay plate are sealed/closed;
Placing and securing the at least one cast with the at least one sealed assay plate in at least one housing,
Descending the at least one housing with the at least one cast with the at least one sealed assay plate to a predetermined location, in particular predetermined depth in a liquid, in particular aqueous environment;
Closing the housing once the predetermined location, in particular predetermined depth is reached, preferably by sending a heavy metal messenger to close the lids of the housing or using sensors for triggering or releasing the closure of the lids;
Moving the at least one sealing plate automatically from the closed position to the open position to open the ports of the wells of the assay plate;
Keeping the ports of the wells open for a predetermined time for allowing bacteria to enter the wells of the assay plate through the ports (e.g. 1 h to 4 h or more);
Moving the at least one sealing plate automatically from the open position to the closed position to close the ports of the wells of the assay plate after the predetermined sampling time; and
Ascending the sampling device, in particular retrieving the sampling device to the surface and the samples are retrieved from the assay plates.

It is to be understood that during descent the at least one housing is open, while the ports of the wells of the assay plates are closed by the sealing plate. Once the predetermined depth is reached the at least one housing is closed. This is done for example by sending down a weight along the wire to which the housing is attached.

The electronics controller of the electronic control unit is triggered automatically when reaching the set depth or desired location and counts down a predetermined amount of buffer time to let turbulence settle in the housing (e.g. 15 min); i.e. a fully autonomous deployment cycle is triggered by depth measurements.

The sampling time, i.e. the time at which the ports of the wells are kept open for bacteria to enter the wells of the assay plate through the ports, is at least 1 hour, preferably at least 2 hours or more.

The chemoattractant compounds that may be employed in the present sampling method can be either single characterized compounds or uncharacterized chemical mixtures, either naturally occurring or man-made. Non exhaustively for example:
Pharmaceuticals (e.g. antibiotics, hormones, analgesics, anti-depressants, anti-inflammatoi anti-parasitics . . . )
Food-derived substances or compounds used in food processing (e.g. additives, stabilizers, preservatives, ethanol, acrylamides . . . )
Chemicals from chemical industry (e.g. dyes, paints, chlorinated solvents, detergents . . . ),
Agriculture-derived chemicals (e.g. pesticides, herbicides, insecticides, fungicides, fertilizers . . . )
Wastewater and environmental water sampled in polluted location
Packaging materials (e.g. plastics, petroleum compounds . . . )
Heavy metals (e.g. lead, cadmium, arsenic, mercury . . . )
Unhealthful molecules and agents (toxins, pathogenic bacterial extracts . . . )

The present sampling method can be divided in three phases: phase A) descent of sampling device; phase B) sampling; phase C) ascent of sampling device.

### Phase A) Descent of Sampling System:

The assay plates are placed in the cast in closed position to avoid any bacterial contamination during descent. The system is secured in the housing whose lids are in open position during the descent to the desired depth. The bottle is attached to a wire.

### Phase B) Sampling:

Once the target depth or desired location is reached, the housing is first closed by sending down a weight along the wire. Some time (e.g. 15 min) is given for the remaining flow in the bottle to settle down and avoid turbulence-induced contamination. The sealing plate automatically moves and exposes the assay plate wells to the surrounding seawater, letting the chemicals diffuse from the wells. The wells are kept open for a predetermined duration to allow the bacteria to enter the wells (usually 1-2 h or more).

### Phase C) Ascent of the Sampling System:

After the predetermined exposure time is ended, the plate slides automatically and seals back the wells, capturing the chemotactic bacteria and making any bacterial contamination during the ascent impossible. The entire system is returned to the surface for sample collection, with the lids of the housing closed to enable sampling of the depth or location-specific water.

### BRIEF DESCRIPTION OF THE DRAWINGS

The terms Fig., Figs., Figure, and Figures are used interchangeably in the specification to refer to the corresponding figures in the drawings.

The solution is explained in more detail by means of examples with reference to the figures.

FIG. 1 is a perspective view of a housing for receiving the cast with assay plates according to the principles of the present disclosure;

FIG. 2 is a perspective view of an assay plate for selectively capturing bacteria of interest according to the principles of the present disclosure;

FIG. 3A is multiple views of a non-limiting embodiment of a cast for holding the assay plate and sealing plate (shown in different angles) according to the principles of the present disclosure;

FIG. 3B is a cross-sectional view of a non-limiting embodiment of a cast for holding the assay plate and sealing plate according to the principles of the present disclosure;

FIG. 4A is multiple views of a non-limiting embodiment of a cast for holding assay plates and sealing plates in combination with electronics, motor and housing according to the principles of the present disclosure;

FIG. 4B is views of a closing mechanism and ground deployment of the housing as shown in FIG. 4A;

FIG. 4C is a view of a deployment of housing from ROV as shown in FIGS. 4A-B;

FIG. 5 is a cross-sectional view of a motor unit according to the principles of the present disclosure;

FIG. 6 is views of a sealing plate for sealing the wells of the assay plate according to the principles of the present disclosure;

FIG. 7 is views of a sealing mechanism of the assay plate using the sealing plate according to the principles of the present disclosure;

FIG. 8 is views of a sampling housing and electronic enclosure according to the principles of the present disclosure;

FIG. 9 is views of a sequence for deploying the sampling device according to the principles of the present disclosure;

FIG. 10 is a diagram showing results of an in situ chemotactic response in a lake at 100 m deep according to the principles of the present disclosure;

FIG. 11 is a diagram showing results of an in situ chemotactic response in the sea at 600 m deep according to the principles of the present disclosure; and

FIG. 12 is a diagram showing results of an in situ chemotactic response in a lake at 6 m deep to environmental pollutants according to the principles of the present disclosure.

### DETAILED DESCRIPTION

### Sampling Device

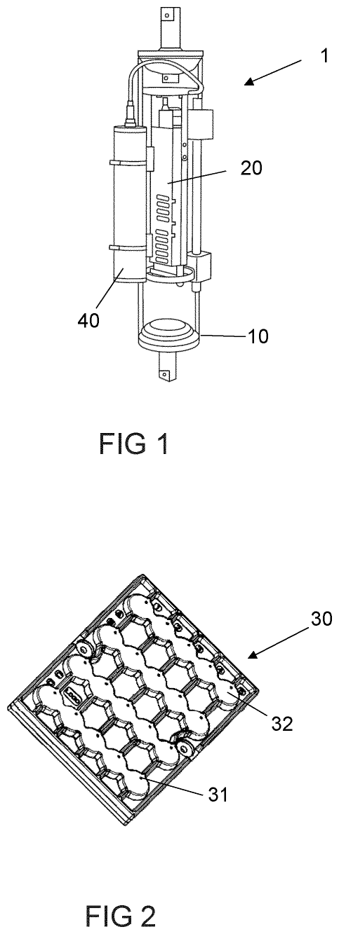

A non-limiting embodiment or aspect of the present sampling device 1 is shown in FIG. 1.

A modified Niskin bottle 10 (10 L, 1150 mm×140 mm) hosting a cast 20 (FIG. 1) is used as housing for receiving up to eight assay plates and two sealing plates and maintaining a low-flow environment during the deployment.

The system was tested in a pressure chamber that guarantees successful functioning down to 4000 m depth.

The cast 20 containing the assay plates and the sealing plates allows deployments at target depths. The sealing plate prevents unwanted contamination during the traveling time through the water column by sealing the ports of the assay plates. At the target depth, the sealing plate opens for a predetermined amount of time and allows for the chemicals to diffuse and chemotaxis to occur, capturing responding bacteria inside the wells of the assay plates.

The electronics housing 40 (outside dimension: 110 mm×322 mm) secured on the side of the bottle allows for a fully stand-alone deployment. The system possesses its own drop weight 17 and needs to be actuated manually.

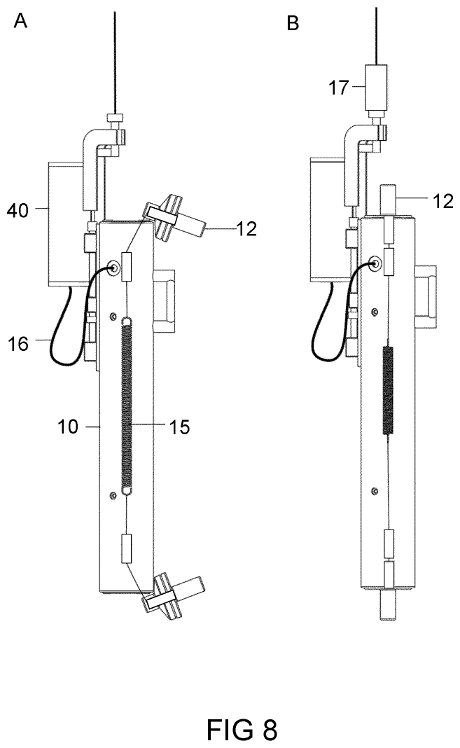

A non-limiting embodiment of the present sampling device is shown in FIG. 8. The electronics housing 40 (outside dimension: 110 mm×322 mm) secured on the side of the housing 10 allows for a fully stand-alone deployment and communicates with the motor unit through a connector cable 16. The bottle is descended to deployment depth with lids open (A). The lids 12 of the housing 10 are maintained open by two springs 15 located on both sides of the housing 10. The system possesses its own drop weight 17 and needs to be actuated manually. Once the drop weight 17 has landed on the activation spring of the housing, the lids 12 close and seal the housing 10 (B).

### Assay Plate for Selectively Capturing Bacteria of Interest

FIG. 2 shows an assay plate 30 suitable for the sampling device and sampling method. The assay plate is a robust and rapidly producible device that bridges the gap between laboratory-based microfluidics and traditional oceanographic methods, by providing an in situ system to interrogate microbial chemotactic behaviors.

The assay plate 30 contains multiple enclosed wells 32. Each (circular) well 32 of the assay plate is enclosed with the exception of a port 31 that allows fluid communication between the inside of each well with the outside environment. Each well has a diameter between 6 and 7 mm. The distance between the center of adjacent wells varies and can be between 9 and 17 mm. The depth of each well is 3 mm.

The assay plate has the size of a credit card (75 mm×47 mm), containing up to twenty wells that can be individually filled with putative chemoattractants (typically, five wells per compound, to ensure enough material for analyses). The device is made of inert material, is single-use and is fabricated using a standard soft lithography workflow, based on a mould created using three-dimensional printing. The assay plate consists of a scalable array of 5×5 wells embedded in a polycarbonate slab, with each well connected to the outside environment, such as seawater or any other environment containing a liquid phase, by the port 31 (0.8 mm diameter, 1.6 mm depth). Most of the wells 32 are filled with chemoattractants, which diffuse out of the ports and into the surrounding seawater during deployment, resulting in chemical microplumes extending 1-2 mm above each well within a typical 1 to 2 h of deployment (or more). The chemical microplumes mimic transient nutrient patches. Chemotactic bacteria respond by swimming into the wells of the device. A row of five wells is always filled with ultrafiltered water originating from the same environment acting as negative control lacking any chemoattractant, that accounts for random microbial motility in the plate.

Chemotactic responses can be quantified by counting bacteria present in the wells using flow cytometry, and the sampled bacteria can be further characterized by extracting their DNA, sequencing them and directly isolating them.

While the original version of the device was made of glass and polydimethylsiloxane (PDMS), the device may also be made of polycarbonate. Polydimethylsiloxane (PDMS) is cast onto a 3D printed mould and cured overnight. The solid PDMS, containing multiple wells, is then excised and plasma-bonded onto a glass slide (100 mm×76 mm×10 mm).

Cast with Sealing Plate and Assay Plate

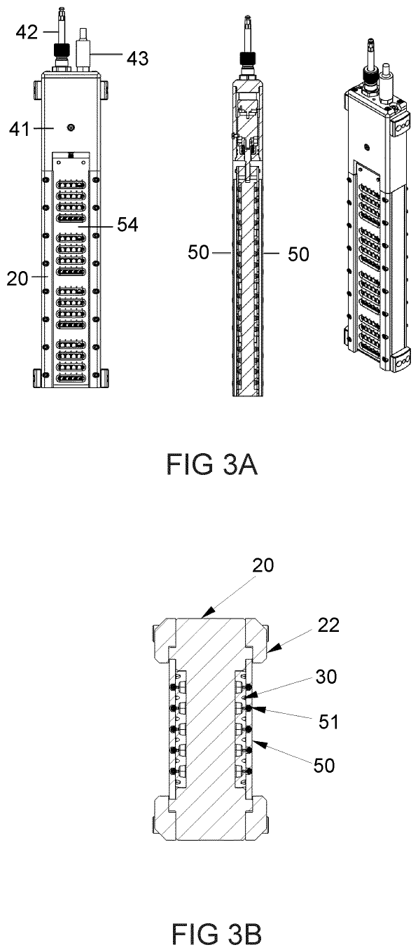

FIG. 3A shows the cast 20, holding the assay plates and containing a pressure compensated motor 41, allowing for autonomous activation at depth (based on a sensor) and a connection 43 to the electronic enclosure 40 (see FIGS. 1, 8). The cast 20 also maintains in place the two sealing plates 50 (500 mm×50 mm), situated on each side of the cast. The rectangular sealing plates 50 seal the ports 31 of the wells 32 of the assay plates with high precision and slides autonomously at the predetermined sampling depth to release the chemical plumes.

The cast 20 (608 mm×56 mm×48 mm) containing the assay plates 30 and the sealing plate 50 allows deployments at target depths. The sealing plate 50 prevents unwanted contamination during the traveling time through the water column by sealing the ports 31 of the assay plates 30. At the target depth, the sealing plate 50 opens for a predetermined amount of time and allows for the chemicals to diffuse and chemotaxis to occur, capturing responding bacteria inside the wells of the assay plates.

The cast 20 and sealing plates 50 are fixed inside the housing (FIGS. 1, 8) and when assembled, enable to access remote environments.

FIG. 3B shows the assay plates 30 mounted on both sides of the cast frame 20. The assay plate carrier allows for location and retention of the assay plates to enable sealing and unsealing by moving the sealing plate 50 over the openings (ports) of the assay plate 30. Pressure rails 22 provide retention of the sealing plates 50 and adequate compression of the spherical sealing elements 51.

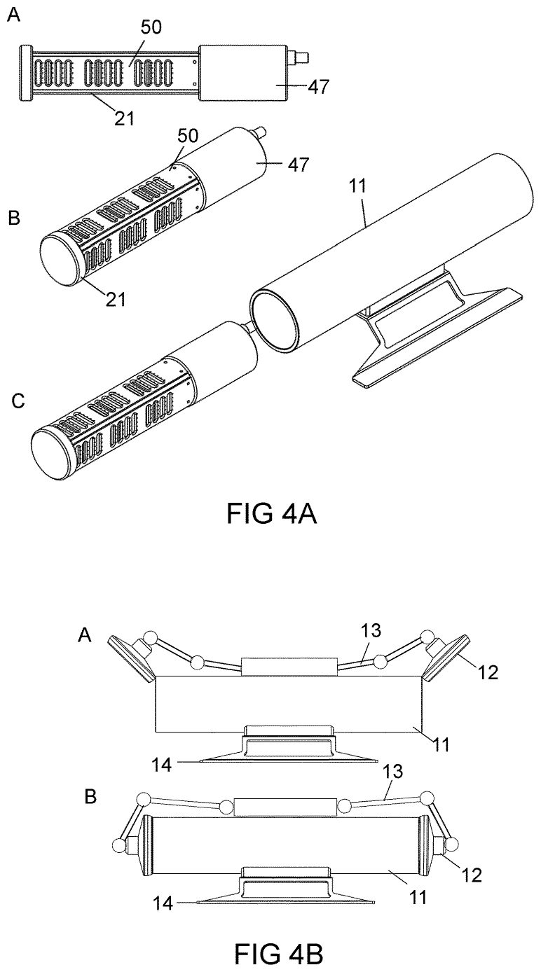

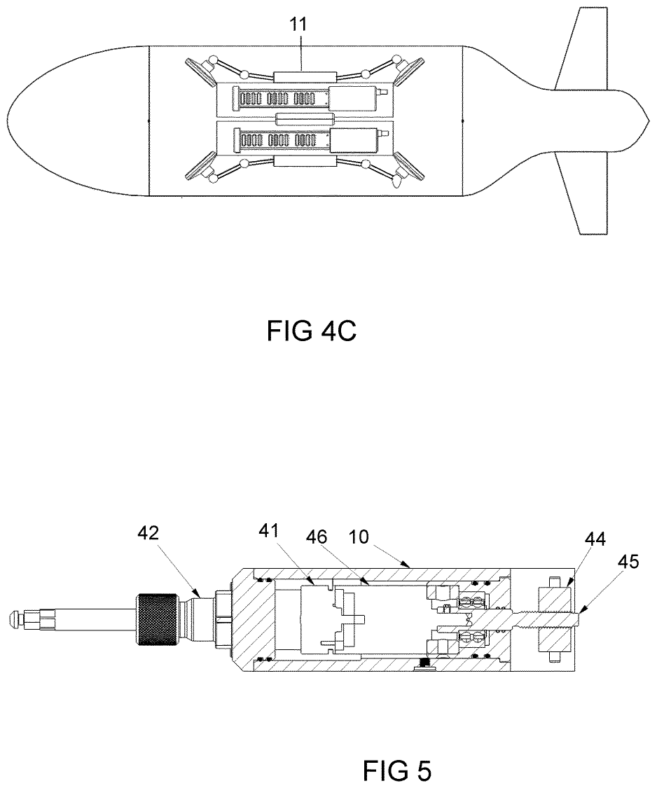

FIG. 4A, B, C show a modification of the cast 20 and housing 10 of the sampling device. In this embodiment, the electronics 40, motor 41 and trigger system are encompassed in a single pressure compensated enclosure 47 connected to the sealing plates 50 and cast 20. This embodiment employs a cubic cast 21 allowing for at least four sealing plates 50 to be actuated at the same time. This system is adjustable in length, allowing to deploy multiples of four assay plates in series (here a total of 12 assay plates are pictured). The pressure compensated enclosure 47 with cubic cast 21 is hosted in a housing 11 protecting the entire system. Here the housing 11 is pictured as standing on a flat surface but can also be in the shape of the housing as shown in FIG. 1.

FIG. 4B shows a non-limiting embodiment of the stand-alone housing 11 with lids 12. In this embodiment, the lids 12 of the housing 11 in open position (A) are closed upon electronic trigger by mechanical arms 13 into a closed position (B). Stabilizing legs 14 are holding the housing on a flat surface. The mechanism enabling the lids 12 of the housing to close would be fully actuated electronically via mechanical arms 13, whereby the entire closing mechanism is controlled by an actuator (and not by springs). This would allow to control the speed, exact timing and closing strength of the lids.

In a non-limiting embodiment the release of the lids 12 of the housing and thereby the closing of the housing would be triggered by retracting a mechanical pin through the help of a solenoid-actuator controlled by the electronics. Once the pin retracted, the lids of the housing would get pulled into their closed position by a set of springs.

FIG. 4C shows a ROV (remotely operated vehicle) housing the sampling devices. The set of legs 14 added to the housing allows the sampling devices to be placed on the sea floor by hand or a ROV. To deploy the system on a ROV of FIG. 4C, the sampling device would be mounted horizontally to fit inside the flooded payload section of a ROV, allowing the deployments to be triggered on specific locations targeted by the ROV, with preference to stationary and undisturbed emplacements. Additionally, the housing could be fitted with features (t-handle, floater knots) allowing for its handling and precise placement by the ROV's manipulator. Note that more than one housing could be deployed by ROV.

FIG. 5 shows the motor unit actuating the sealing plate for sealing and opening the wells of the assay plate. An electric motor 41 with a reducing gear box 46 connected to a lead screw 45 drives a nut 44 to move the sealing plate in a linear motion at slow speed and high force. The motor, gear box and bearing are mounted inside a housing which is filled with dielectric oil to protect the components from the surrounding water during deployment. The housing is pressure compensated with a pressure compensator 42 to allow for operation at depth.

### Sealing Plate

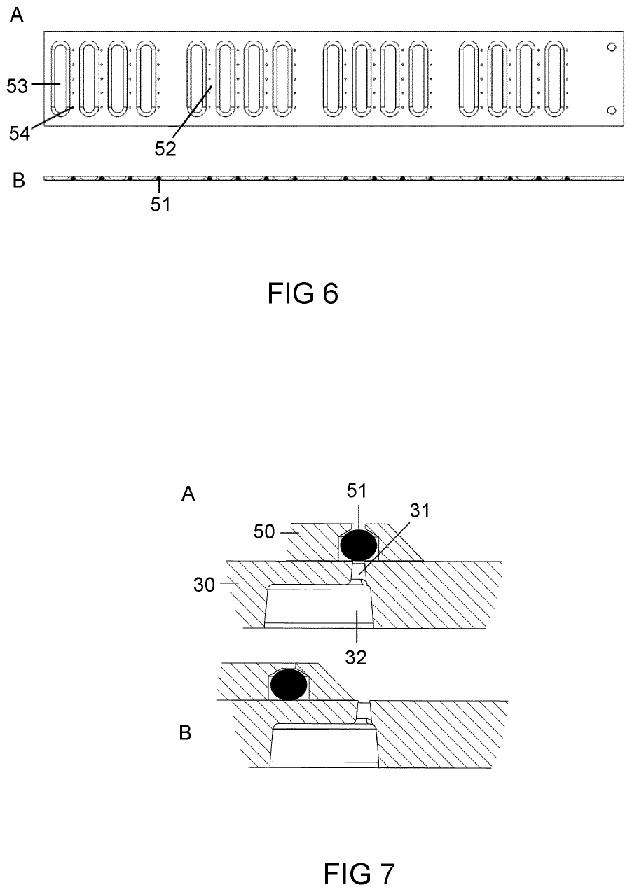

FIG. 6 shows a sealing plate 50 in top view (A) and in cross-section (B). The sealing plate 50 has the shape of a grid with alternating slit-forming gaps 53 for open position and bridges 52 for sealed position. The bridges 52 are provided with evenly spaced rubber spheres 51 as means for sealing the ports 31 of the assay plate wells 32. The rubber spheres are embedded in the plate within small niches 54 and provide an efficient seal while in the closed position.

The number of rubber spheres on each bridge is identical to the number of ports in each row of the assay plate.

FIG. 7 shows the sealing mechanism of the assay plate 30 by the rubber spheres 51 embedded in the sealing plate 50. In closed position (A), the rubber spheres 51 are positioned on top of the assay plate ports 31 and seal them to prevent contamination and chemical leakage from the wells 32. Once triggered, the sealing plate 50 opens (B) and exposes the port 31 of the wells 32 to the external environment.

### Sampling Method

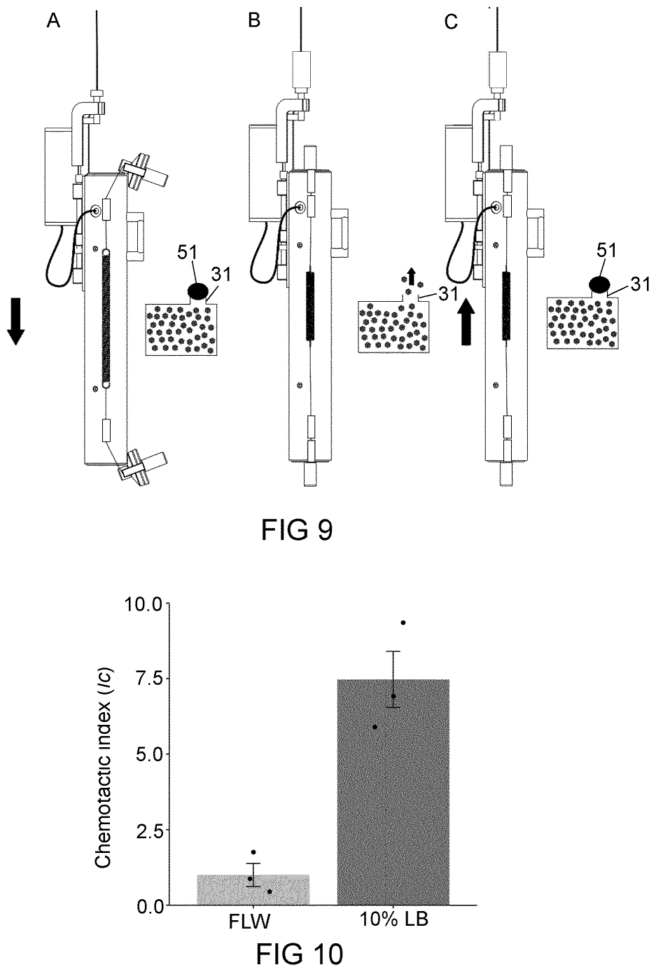

FIG. 9 illustrates the sequence of deployment of the sampling device. The deployment can be divided in three phases: phase A) descent of sampling device; phase B) sampling; phase C) ascent of sampling device.

Descent of the sampling system to predetermined depth (FIG. 9A.):
1. The assay plates are placed in the cast in closed position to avoid any bacterial contamination during descent. The well ports 31 of the assay plate 30 are covered and sealed by the rubber spheres 51 of the sealing plate 50.
2. The system is secured in the housing, whose lids are in open position during the descent to the desired depth.
3. The housing is attached to a wire.

Sampling at predetermined depth: (FIG. 9B)
1. Once the target depth is reached, the housing is first closed by sending down a weight along the wire.
2. Some time (e.g. 15 min) is given for the remaining flow in the bottle to settle down and avoid turbulence-induced contamination.
3. The sealing plate automatically moves and exposes the assay plate wells to the surrounding seawater, letting the chemicals diffuse from the wells. The well ports 31 of the assay plates 30 are now open.
4. The wells are kept open for a predetermined duration to allow the bacteria to enter the wells (usually 1-2 h, or more).

Ascent of the sampling system (FIG. 9C)
1. After the predetermined exposure time is ended, the sealing plate slides automatically and seals back the wells, capturing the chemotactic bacteria and making any bacterial contamination during the ascent impossible. The well ports 31 of the assay plates 30 are again sealed by the rubber spheres 51 of the sealing plate 50.
2. The entire system is returned to the surface for sample collection, with the lids of the housing closed to enable sampling of the depth or location-specific seawater.

FIG. 10 shows a diagram illustrating the in situ chemotactic response of a natural lake microbial population at depth (100 m) using the sampling device toward a rich medium (10% Luria-Bertani broth) after 60 min of deployment. The chemotaxis index I_{C} represents the strength of attraction of the bacterial community towards a compound compared to a negative control of ultrafiltered lake water lacking any chemoattractant, and therefore accounting only for random swimming of bacteria in the device. The I_{C} is calculated by normalizing the cell counts obtained in the treatment wells by the mean cell count within the wells containing the filtered lake water negative controls (FLW). Each treatment and control were tested in three assay plate replicates contained in the deployment system. Bacterial cells were quantified by flow cytometry (FLW=5.73±3.79×10³; 10% LB=4.28±0.92×10⁴ cells/mL). The concentration of LB medium tested at lake depth induced a chemotactic response significantly higher than the filtered lake water (FLW) controls. In all pairwise comparisons: t-test, p<0.005; Error bars represent SEM.

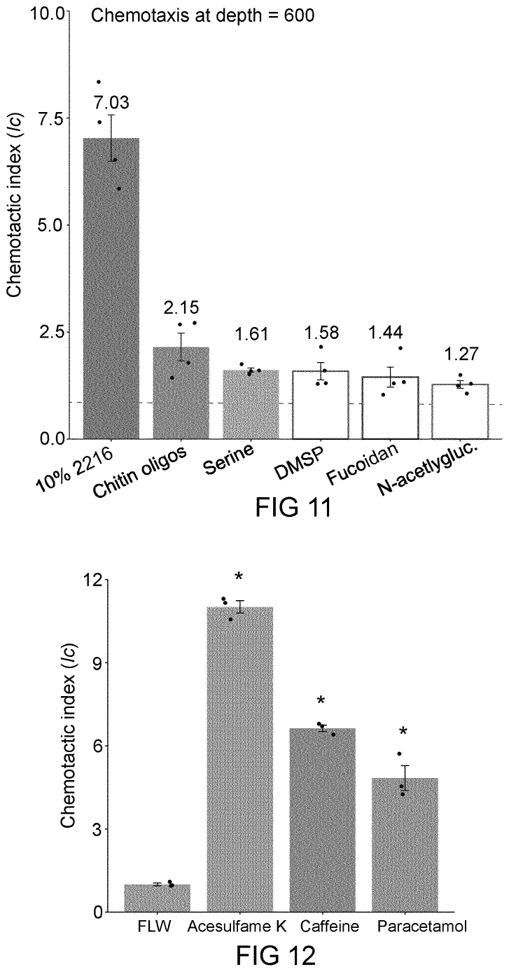

FIG. 11 shows a diagram illustrating the in situ chemotactic response of a natural marine microbial population at depth (600 m) after 2 h of deployment using the same sampling device toward highly characterized bacterial chemoattractants (10% Marine Broth 2216, 1 mM serine and 1 mM DMSP) and natural constituents of marine nutrient patches and particles (1 mM N-acetylglucosamine, 10 mg/ml fucoidan and 10 mg/ml chitin oligosaccharides). The chemotactic index I_{C} represents the strength of attraction of the bacteria community towards a compound compared to a negative control of ultrafiltered seawater lacking any chemoattractant, and therefore accounting only for random swimming of bacteria in the device. The I_{C} is calculated by normalizing the cell counts obtained in the treatment wells by the mean cell count within the wells containing the filtered seawater negative controls (FSW). Each treatment and control were tested in four assay plate replicates contained in the deployment system. Bacterial cells were quantified by flow cytometry (FSW=1.34±0.30×10⁴; 10% MB2216=9.4±1.45×10⁴; serine=2.16±0.13×10⁴; DMSP=2.12±0.54×10⁴; N-acetylglucosamine=1.7±0.23×10⁴; fucoidan=1.94±0.63×10⁴; chitin oligosaccharides=2.88±0.86×10⁴ cells/mL). The concentration of Marine Broth 2216, chitin oligosaccharides and serine deployed at 600 m deep in the sea induced a chemotactic response significantly higher than the filtered seawater (FSW) controls, of I_{C}=7.03, 2.15 and 1.61 respectively. In all pairwise comparisons: t-test, p<0.005; Error bars represent SEM.

FIG. 12 shows a diagram illustrating the in situ chemotactic response of a natural lake microbial population at depth (5 m) using the sampling device toward three emerging pollutants (acesulfame K, caffeine and paracetamol) after 60 min of deployment. The chemotaxis index I_{C} represents the strength of attraction of the bacterial community towards a compound compared to a negative control of ultrafiltered lake water lacking any chemoattractant, and therefore accounting only for random swimming of bacteria in the device. The I_{C} is calculated by normalizing the cell counts obtained in the treatment wells by the mean cell count within the wells containing the filtered lake water negative controls (FLW). Each treatment and control were tested in three assay plate replicates contained in the deployment system. Bacterial cells were quantified by flow cytometry (FLW=1.44±0.12×10⁴; acesulfame K=1.59±0.55×10⁵; caffeine=9.56±0.29×10⁴; paracetamol=6.98+0.12×10⁴ cells/mL). The concentration of all three man-made compounds tested at lake depth induced a chemotactic response significantly higher than the filtered lake water (FLW) controls. In all pairwise comparisons: t-test, p<0.005; Error bars represent SEM.

1. A sampling device for collecting microorganisms from an environment by chemotaxis comprising:
a housing configured to receive at least one cast,
wherein the at least one cast comprises means that are configured to receive and hold at least one assay plate,
wherein the at least one assay plate comprises at least one enclosed well filled with at least one chemoattractant compound and at least one water negative control, and
wherein each enclosed well comprises at least a port configured for fluid communication between the inside and the outside of the enclosed well;
wherein the cast is further configured to receive at least one sealing plate at least partially covering the at least one assay plate,
wherein the sealing plate comprises at least one sealing element for sealing the port of at least one enclosed well of the assay plate,
wherein the sealing plate is movably mounted on the cast, such that it at least partially covers the at least one assay plate held by the cast,
wherein said sealing plate is movable between
a first position, wherein the at least one sealing plate seals the port of the at least one well of the assay plate (sealing position/closed position), and
a second position, wherein the at least one sealing plate opens the port of the at least one well of the assay plate (open position)

2. The sampling device according to claim 1, wherein the at least one cast is made of pressure and corrosion resistant material.

3. The sampling device according to claim 1, wherein the at least one assay plate contains 20 wells or more that each can be filled with at least one chemoattractant compound.

4. The sampling device according to claim 1, wherein the sealing plate is designed in a grid-like manner with alternating slits-forming gaps for open position and bridges for sealed position.

5. The sampling device according to claim 1, wherein the at least one sealing plate is provided with elements for sealing the ports of each well of the assay plate.

6. The sampling device according to claim 5, wherein the sealing elements are arranged on the bridges of the sealing plate.

7. The sampling device according to claim 5, wherein elements for sealing the ports of each well are evenly spaced for aligning with the evenly spaced port of the wells of the assay plate.

8. The sampling device according to claim 5, wherein the at least one sealing element is designed in a spheric shape.

9. The sampling device according to claim 1, wherein the cast further contains at least one motor for moving the at least one sealing plate along the at least one assay plate between the first position and the second position and conversely.

10. The sampling device according to claim 1, wherein the at least one electronic control unit is secured on the side of the housing in a titanium enclosure allowing for a fully stand-alone deployment.

11. A method for collecting bacteria from an aquatic environment by chemotaxis employing a sampling device according to claim 1, wherein the method comprises:
providing at least one assay plate with at least one well filled with at least one chemoattractant compound;
placing the at least one assay plate in the at least one cast, wherein the at least one assay plate is at least partially covered by the at least one sealing plate such that the ports of the wells of the assay plate are sealed/closed;
placing and securing the at least one cast with the at least one sealed assay plate in at least one housing;
Descending the at least one housing with the at least one cast with the at least one sealed assay plate to a predetermined location in a liquid;
closing the housing once the predetermined location is reached,
moving the at least one sealing plate automatically from the closed position to the open position to open the ports of the wells of the assay plate;
keeping the ports of the wells open for a predetermined time for allowing bacteria to enter the wells of the assay plate through the ports;
moving the at least one sealing plate automatically from the open position to the closed position to close the ports of the wells of the assay plate after the predetermined sampling time; and
ascending the sampling device.

12. The method according to claim 11, wherein during descent the at least one housing is open, while the ports of the wells of the assay plates are closed by the sealing plate.

13. The method according to claim 11, wherein the at least one housing is closed once the predetermined depth is reached.

14. The method according to claim 11, wherein the sampling time is at least 1 hour.

15. The method according to claim 11, wherein the sampling device is deployed in any type of environment containing at least an aqueous phase.

16. The sampling device according to claim 1, wherein the environment is an aqueous environment.

17. The sampling device according to claim 2, wherein the at least one cast is made of polyoxymethylene (POM).

18. The sampling device according to claim 8, wherein the at least one sealing element is designed in the form of a rubber sphere.

19. The sampling device according to claim 9, wherein at least one motor is a pressure compensated motor.

20. The method according to claim 11, wherein the at least one housing is a modified Niskin bottle.
